(19) United States
(12) Patent Application Publication
(10) Pub. No.: US 2026/0174954 A1
(43) Pub. Date: Jun. 25, 2026

(54) CONTEXTUAL INPUT SYSTEM FOR AUTOMATED INSULIN DELIVERY

(71) Applicant: QUASURAS, INC., San Diego, CA (US)

(72) Inventor: Paul M. DiPerna, Escondido, CA (US)

(21) Appl. No.: 19/429,216

(22) Filed: Dec. 22, 2025

Related U.S. Application Data
(60) Provisional application No. 63/738,420, filed on Dec. 23, 2024.

(51) Int. Cl. A61M 5/142

(52) U.S. Cl. CPC A61M 5/14248; A61M 2005/14208; A61M 2202/0007; A61M 2202/04; A61M 2205/3303; A61M 2205/3327; A61M 2205/3334; A61M 2205/3553; A61M 2205/3592; A61M 2205/502; A61M 2205/583; A61M 2230/005; A61M 2230/201

(57) ABSTRACT

An insulin delivery system for diabetes management includes a patch-based insulin pump configured to deliver insulin to a user, a user input button disposed on the patch-based insulin pump, a processor configured to execute a hybrid closed loop algorithm that automatically controls insulin delivery based on glucose levels. The user input button is configured to receive user input comprising a sequence of button presses that communicate contextual information about user activities to the hybrid closed loop algorithm, the contextual information being distinct from direct insulin bolus delivery commands. The contextual information may include meal intake, physical activity, and sleep status communicated through different sequences of button presses. The system includes confirmation mechanisms that provide feedback and allow cancellation of inputs, enabling discrete real-time communication of personalized data to enhance diabetes management.

### CROSS-REFERENCE TO RELATED APPLICATIONS

This application claims priority to U.S. Provisional Patent Application No. 63/738,420 filed on December 23, 2024 and titled “QUICK BOLUS BUTTON REUSE” then entirety of which is incorporated by reference herein.

### FIELD OF INVENTION

The present disclosure relates to automated insulin delivery systems for diabetes management, and more particularly to a contextual input system that uses a user input button to communicate user activity information to hybrid closed loop algorithms for improved insulin dosing control.

### BACKGROUND

People with diabetes rely on insulin therapy to manage their blood glucose levels and maintain health. Traditional insulin delivery methods, such as multiple daily injections, provide limited flexibility and often result in suboptimal glucose control. Insulin pumps have emerged as an advanced therapeutic option, offering more precise insulin delivery through programmable basal rates and on-demand bolus doses. The bolus button has been used for over 25 years in a variety of insulin pump products to allow users to directly instruct the pump to deliver bolus insulin doses.

Modern insulin pump systems have evolved to incorporate continuous glucose monitoring technology, enabling the development of hybrid closed loop systems. These systems automatically adjust insulin delivery based on real-time glucose readings, reducing the burden of diabetes management on users. The term 'hybrid closed loop' refers to a semi-automated insulin delivery system that automatically adjusts basal insulin delivery based on continuous glucose monitoring data, but still requires user input for certain functions such as meal announcements or bolus delivery. This is distinguished from a fully closed loop or 'artificial pancreas' system that would require no user input. The 'hybrid' nature acknowledges that user contextual information enhances algorithmic performance beyond what can be achieved through glucose data alone. Insulin therapy has evolved with pumping systems having internal algorithms that review blood glucose levels from continuous glucose measuring systems to calculate and automate the delivery of insulin. However, hybrid closed loop algorithms face challenges in accurately predicting insulin needs during various daily activities and physiological states. Since the body can very rapidly uptake glucose at meals and is relatively slow on the uptake of insulin delivered subcutaneously, people have continued to use the button to deliver bolus insulin to prevent spiking high glucose after meals as the action of insulin tries to catch up.

Current insulin pump interfaces typically rely on complex menu systems and multiple button configurations to allow users to input information about meals, exercise, and other factors that affect glucose levels. Traditional input methods require users to open an app on their phone, navigate to the input screen, and indicate the desired input of information. These interfaces can be cumbersome and may discourage users from providing the contextual information that would improve algorithmic decision-making. The complexity of existing input methods often leads to underutilization of available features, potentially compromising glucose control outcomes.

The effectiveness of automated insulin delivery systems depends heavily on the algorithm's ability to anticipate and respond to changes in insulin requirements throughout the day. Factors such as meal consumption, physical activity, sleep patterns, and stress can significantly impact glucose levels and insulin sensitivity. Without adequate user input about these contextual factors, even sophisticated algorithms may struggle to provide optimal insulin dosing. Above range glucose levels are presently estimated to be approximately 30% of the time, indicating significant room for improvement in algorithmic performance.

There exists a need for simplified input mechanisms that encourage user engagement while providing automated insulin delivery systems with the contextual information necessary for improved glucose management. Such systems would benefit from intuitive interfaces that seamlessly integrate user activity information into algorithmic decision-making processes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In one aspect, an insulin delivery system for diabetes management includes a patch-based insulin pump configured to deliver insulin to a user, a user input button disposed on the patch-based insulin pump, and a processor configured to execute a hybrid closed loop algorithm that automatically controls insulin delivery based on glucose levels. The user input button is configured to receive user input comprising a sequence of button presses that communicate contextual information about user activities to the hybrid closed loop algorithm, the contextual information being distinct from direct insulin bolus delivery commands.

Additionally or alternatively, the insulin delivery system further comprises a glucose sensor in communication with the processor configured to provide the processor with glucose level data.

Additionally or alternatively, the processor is a remote computing system in wireless communication with the patch-based insulin pump, the user input button, and the glucose sensor.

Additionally or alternatively, the contextual information comprises meal information indicating a size of a meal being consumed by the user, wherein the meal information is communicated through a first number of button presses corresponding to small, medium, or large meal sizes.

Additionally or alternatively, the contextual information comprises physical activity information indicating upcoming exercise by the user, wherein the physical activity information is communicated through a second number of button presses indicating a duration until the exercise begins.

Additionally or alternatively, the contextual information comprises sleep information indicating bedtime or wake-up time of the user.

Additionally or alternatively, the insulin delivery system further comprises a confirmation mechanism that provides feedback to the user after receiving the sequence of button presses.

In another aspect, a method of managing diabetes using an automated insulin delivery system comprises providing a patch-based insulin pump having a user input button and executing a hybrid closed loop algorithm, receiving a sequence of button presses from the user input button, wherein the sequence of button presses communicates contextual information about user activities selected from the group consisting of meal intake information, physical activity information, and sleep status information, processing the contextual information with the hybrid closed loop algorithm, and automatically adjusting insulin delivery based on the contextual information and glucose level data.

Additionally or alternatively, the meal intake information is communicated through a first sequence of button presses corresponding to small, medium, or large meal sizes.

Additionally or alternatively, the method further comprises a step of providing visual feedback to confirm receipt of the meal intake information before processing by the hybrid closed loop algorithm.

Additionally or alternatively, the physical activity information is communicated through a second sequence of button presses indicating a duration until exercise begins.

Additionally or alternatively, automatically adjusting insulin delivery comprises reducing insulin delivery or raising a target glucose level for a predetermined time period before the indicated exercise.

Additionally or alternatively, the method further comprises a step of requiring user confirmation by holding the user input button for a first predetermined time period after receiving the sequence of button presses.

Additionally or alternatively, the method further comprises a step of canceling the contextual information input when the user input button is held for a second predetermined time period, wherein the second predetermined time period is longer than the first predetermined time period.

In another aspect, an insulin delivery system for diabetes management includes a patch-based insulin pump, a user input button disposed on the patch-based insulin pump, and a processor configured to execute a hybrid closed loop algorithm that automatically controls insulin delivery based on glucose levels. The user input button is configured to receive different sequences of button presses corresponding to different types of contextual information about user activities, and the processor is configured to modify operation of the hybrid closed loop algorithm based on the contextual information communicated through the different sequences of button presses.

Additionally or alternatively, the different types of contextual information comprise meal intake information, physical activity information, and sleep status information.

Additionally or alternatively, the insulin delivery system further comprises a feedback mechanism that provides visual feedback to indicate which type of contextual information is being received.

Additionally or alternatively, the insulin delivery system further comprises a confirmation mechanism that requires the user to hold the user input button for a first predetermined time period to confirm the contextual information being communicated to the hybrid closed loop algorithm, and wherein the confirmation mechanism is configured to cancel the contextual information input when the user input button is held for a second predetermined time period, wherein the second predetermined time period is longer than the first predetermined time period.

Additionally or alternatively, the processor is configured to distinguish between the different sequences of button presses based on timing intervals between individual button presses within each sequence.

Additionally or alternatively, the hybrid closed loop algorithm is configured to adjust at least one of basal insulin delivery rate or target glucose level in response to receiving the contextual information.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a system diagram of an automated insulin delivery system for diabetes management, according to aspects of the present disclosure.

FIG. 2 illustrates a block diagram of a contextual processing system for the automated insulin delivery system of FIG. 1, according to aspects of the present disclosure.

FIG. 3 illustrates a block diagram of a contextual information classification system for processing user input data, according to aspects of the present disclosure.

FIG. 4 illustrates a flowchart for a method of processing multiple types of contextual information in the automated insulin delivery system, according to aspects of the present disclosure.

FIG. 5 illustrates a flowchart for a method of processing meal size information in the automated insulin delivery system, according to aspects of the present disclosure.

FIG. 6 illustrates a flowchart for a method of processing physical activity information in the automated insulin delivery system, according to aspects of the present disclosure.

FIG. 7 illustrates a flowchart for a method of processing contextual information with user confirmation, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure addresses limitations in existing automated insulin delivery systems by introducing a simplified contextual input mechanism that enhances hybrid closed loop algorithm performance without requiring complex user interfaces. Traditional insulin pump systems rely on cumbersome menu navigation and multiple button configurations to capture user activity information, which often leads to underutilization and suboptimal glucose control. The disclosed system overcomes these challenges through several inventive concepts.

The primary inventive concept involves using a user input button to communicate contextual information about user activities through distinct sequences of button presses. Unlike conventional systems that require direct insulin bolus commands or extensive menu interaction, this approach enables users to convey meaningful activity data—such as meal intake, physical activity timing, and sleep status—through simple, intuitive button press patterns. This distinction between contextual information and direct dosing commands represents a departure from traditional pump interfaces.

A second inventive concept relates to the encoding of different types and magnitudes of contextual information through variations in button press sequences. The system may distinguish between small, medium, and large meals through different numbers of button presses, and may communicate timing information for upcoming exercise through additional press patterns. This multi-dimensional encoding capability allows a single button to replace multiple interface elements while maintaining specificity in the information conveyed.

The disclosure further introduces confirmation and cancellation mechanisms that provide user feedback and error correction without additional interface complexity. By implementing time-based button hold functions with different durations for confirmation versus cancellation, the system ensures accurate data transmission while maintaining interface simplicity.

The technical advantages of these inventive concepts include improved user engagement through reduced interface complexity, enhanced algorithm performance through better contextual awareness, and more precise insulin delivery adjustments based on anticipated physiological changes. The system enables real-time communication of personalized activity data that allows the hybrid closed loop algorithm to proactively adjust basal rates and target glucose levels, rather than reactively responding to glucose excursions after they occur. This proactive capability may reduce glucose variability and improve overall diabetes management outcomes while minimizing the cognitive burden on users.

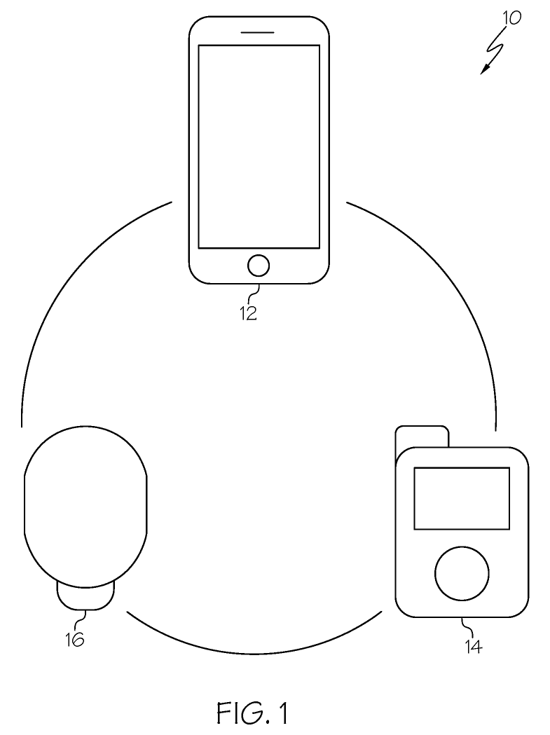

Referring to FIG. 1, an insulin delivery system for diabetes management 10 is illustrated in a system diagram showing the interconnected components and their communication pathways. The system 10 comprises a processor 12, which in some aspects may be implemented as a remote computing device such as a mobile phone, computer, smartphone, tablet, or dedicated controller. The processor 12 is configured to execute a hybrid closed loop algorithm that automatically controls insulin delivery based on glucose levels and contextual information received from user inputs.

The system 10 further includes an insulin pump 14, which in some aspects may be configured as a patch-based device that adheres to the user's body for insulin delivery. As used herein, a 'patch-based insulin pump' refers to an insulin delivery device that adheres directly to the user's skin, typically through an adhesive backing, and integrates the insulin reservoir, pumping mechanism, and infusion set in a compact, self-contained unit. Patch-based pumps may be distinguished from traditional pumps that use separate infusion sets connected by tubing. The patch-based configuration allows for discrete, tubeless insulin delivery that can be worn under clothing. The insulin pump 14 is in wireless communication with the processor 12, allowing the processor 12 to transmit insulin delivery commands based on algorithmic calculations. A user input button is disposed on the insulin pump 14, though in some configurations the button may be integrated with the processor 12 or provided as a separate input device. The user input button is configured to receive user input comprising a sequence of button presses that communicate contextual information about user activities to the hybrid closed loop algorithm executing on the processor 12. In some aspects, the system may automatically detect the type of contextual information being communicated based on the number and timing of button presses, without requiring the user to explicitly select an input mode. In other aspects, the system may cycle through different input modes indicated by different LED colors, and the user may select a desired mode by beginning their button press sequence when the appropriate color is displayed. In some aspects, the user input button may also serve additional functions, such as a priming function during initial assembly of the pump and cartridge, where a button push tells the pump to prime by pumping fluid until a drop is detected at the end of the tubing. During normal operation, a single press may wake the system up, confirm it is working, and send information to a phone app, allowing the user to determine what is going on with the system.

A glucose sensor 16 is also shown in communication with the processor 12. The glucose sensor 16 may be configured as a continuous glucose monitoring device that measures interstitial glucose levels and transmits glucose level data to the processor 12 at regular intervals. In some aspects, the glucose sensor 16 may transmit data wirelessly using protocols such as Bluetooth Low Energy or other suitable wireless communication standards. In some aspects, the glucose sensor 16 may transmit glucose level data at regular intervals, such as every 1 minute, every 5 minutes, or every 15 minutes, depending on the specific sensor technology and system configuration. The glucose level data provided by the glucose sensor 16 serves as a primary input to the hybrid closed loop algorithm, enabling the processor 12 to make informed decisions about insulin delivery adjustments. The components of the hybrid closed loop system, including the processor 12, insulin pump 14, and glucose sensor 16, work together to provide semi-automated diabetes management that responds to glucose quantities in the blood.

The circular arrangement of components in FIG. 1 illustrates the bidirectional flow of information within the system 10. The processor 12 receives glucose data from the glucose sensor 16 and contextual information from the user input button, processes this information through the hybrid closed loop algorithm, and transmits control signals to the insulin pump 14 to adjust insulin delivery accordingly. In some aspects, the processor 12 may also provide feedback to the user through visual displays, audible alerts, or haptic feedback mechanisms such as sound or vibration to confirm receipt of button press sequences or to communicate system status information. The feedback mechanisms described herein, including LED lights, sound, and vibration, may be provided individually or in combination depending on the specific implementation. In some aspects, the pump may include an LED display that provides visual feedback through different colors or patterns. In other aspects, the pump may provide audible feedback through a speaker or piezoelectric element, or haptic feedback through a vibration motor. When the system 'confirms with a repeat of the button presses,' this may mean that the system generates the same number of beeps, vibrations, or light flashes as the number of button presses received, allowing the user to verify that the input was correctly detected. The button on the patch pump system attached to the body allows the user to discretely input information to the algorithm in real-time, enhancing their diabetes management without requiring complex menu navigation or opening apps on external devices.

The wireless communication architecture shown in FIG. 1 enables the system 10 to operate as an integrated diabetes management platform where multiple data sources inform algorithmic decision-making. In some cases, the processor 12 may store historical glucose data, insulin delivery records, and contextual information inputs to enable trend analysis and predictive modeling. In some aspects, the processor 12 may store historical glucose data, insulin delivery records, and contextual information inputs in local memory or in cloud-based storage systems. The historical data may be retained for periods ranging from several days to several years, enabling long-term trend analysis, pattern recognition, and personalized algorithm optimization. The stored data may also be accessible to healthcare providers for remote monitoring and therapy adjustment. The system 10 may also be configured to communicate with additional devices or cloud-based services for data backup, remote monitoring by healthcare providers, or integration with other health tracking applications.

The system 10 represents a departure from traditional button-based insulin delivery systems. Rather than using the button to directly instruct the pump to deliver bolus insulin doses, the disclosed system 10 redeploys the button to inform the algorithm of what is going on with the user. The contextual information communicated through button press sequences is distinct from direct insulin bolus delivery commands in that the contextual information informs the hybrid closed loop algorithm about user activities and physiological states, allowing the algorithm to automatically calculate and adjust insulin delivery parameters, rather than directly instructing the pump to deliver a specific quantity of insulin immediately. In some aspects, the system may eliminate direct bolus button functionality entirely, relying instead on the algorithm to determine all insulin delivery based on glucose levels and contextual information. In other aspects, the system may retain separate mechanisms for direct bolus delivery while using the button press sequences described herein exclusively for contextual information input. This approach frees up the button from bolus obligations and allows for a variety of alternative inputs to better manage the algorithm and the user's condition. The button press inputs provide contextual information to the algorithm to better model what is going on with the user, allowing the insulin dosing to be more aggressive in reducing above range glucose levels. This discrete input capability enables users to provide personalized data in real-time without the need to access external devices or navigate through complex app interfaces.

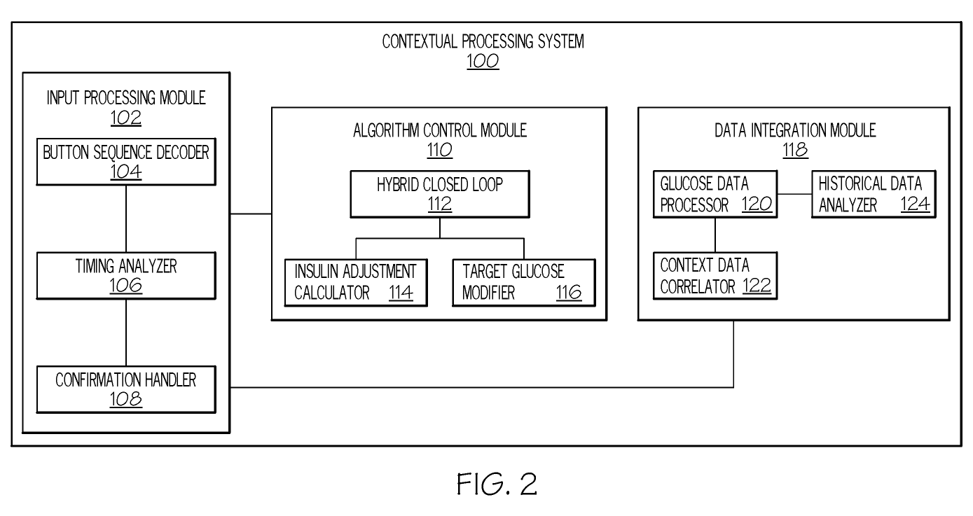

Referring to FIG. 2, a contextual processing system 100 is illustrated in a block diagram showing an example of functional modules for processing user input data in the automated insulin delivery system. In an exemplary embodiment, the contextual processing system 100 comprises three primary modules that work together to convert button press sequences into actionable control parameters for insulin delivery management. It should be understood that the contextual processing system 100 shown may be one exemplary modular system, but that the invention may be embodied by any system which performs the functionality described herein. The processor 12 and the various processing modules described herein (such as the contextual processing system 100 and contextual information classification system 200) may be implemented entirely within the remote computing device, entirely within the insulin pump 14, or distributed between multiple devices in communication with each other. In some aspects, the processor 12 executing the hybrid closed loop algorithm may reside in a mobile phone or computer, while the button sequence decoder 104 and timing analyzer 106 may reside in the insulin pump 14, with the decoded contextual information being transmitted wirelessly to the processor 12 for algorithmic processing.

In the embodiment shown, the input processing module 102 receives and interprets user input from the button interface disposed on the insulin pump 14. Within the input processing module 102, the button sequence decoder 104 is configured to decode sequences of button presses received from the user input button. The button sequence decoder 104 analyzes the pattern of button presses to determine what type of contextual information the user is attempting to communicate. In some aspects, button presses may be done in one second increments, with the system analyzing the timing between presses to distinguish between different input types. In some aspects, the system may allow a tolerance window of plus or minus 0.5 seconds for each button press interval, such that intervals between 0.5 and 1.5 seconds are recognized as valid one-second increments. The timing analyzer 106 may use these timing intervals to distinguish between rapid successive presses intended as a count versus longer pauses that indicate the end of a sequence. The processor 12 is configured to distinguish between the different sequences of button presses based on timing intervals between individual button presses within each sequence. The decoded information is transmitted to the timing analyzer 106, which examines the temporal characteristics of the button press sequence, including the intervals between individual presses and the overall duration of the sequence. The timing analyzer 106 may distinguish between different types of contextual information based on these timing parameters, enabling the system to differentiate between meal information, physical activity information, and sleep status information using a single button interface. The processed timing data is then transmitted to the confirmation handler 108, which manages the confirmation and validation process before the contextual information is transmitted to other system components. In some aspects, the confirmation handler 108 may require the user to hold the button for 3-5 seconds to confirm the information being sent to the algorithm, or may detect a 10 second press to cancel the input if necessary.

The algorithm control module 110 receives validated contextual information from the confirmation handler 108 and manages the operation of the hybrid closed loop algorithm. The hybrid closed loop 112 serves as the central control component that automatically adjusts insulin delivery based on glucose levels and the contextual information provided by users. The hybrid closed loop algorithm 112 is configured to adjust at least one of basal insulin delivery rate or target glucose level in response to receiving the contextual information. The hybrid closed loop 112 transmits control signals to two parallel processing components: the insulin adjustment calculator 114 and the target glucose modifier 116. The insulin adjustment calculator 114 calculates modifications to basal insulin delivery rates based on the contextual information received, determining whether insulin delivery should be increased, decreased, or maintained at current levels. The target glucose modifier 116 operates in parallel to adjust target glucose levels in response to anticipated physiological changes indicated by the contextual information. In some aspects, the target glucose modifier 116 may raise target glucose levels in anticipation of physical activity to reduce the risk of exercise-induced hypoglycemia, or may adjust targets during sleep periods to optimize overnight glucose control.

The data integration module 118 processes and correlates multiple data sources to provide comprehensive information to the algorithm control module 110. The glucose data processor 120 receives continuous glucose level data from the glucose sensor 16 and processes this data to extract relevant metrics such as current glucose level, rate of change, and trend direction. The processed glucose data is transmitted to both the context data correlator 122 and the historical data analyzer 124. The historical data analyzer 124 maintains and analyzes historical records of glucose data, insulin delivery patterns, and contextual information inputs to identify trends and patterns that may inform future algorithmic decisions. The context data correlator 122 receives inputs from both the glucose data processor 120 and the historical data analyzer 124, and correlates current glucose data with contextual information to provide integrated data to the hybrid closed loop 112. This correlation enables the algorithm control module 110 to make more informed decisions by considering both current physiological state and anticipated changes based on user activities.

The hybrid closed loop algorithm may respond to contextual information inputs with varying magnitudes and durations of adjustment based on the specific information received. For example, upon receiving a large meal indication, the algorithm may increase basal insulin delivery by 20-50% for a period of 2-4 hours, or may calculate and deliver a meal bolus based on the estimated carbohydrate content of a large meal. Upon receiving a medium meal indication, the algorithm may increase basal insulin delivery by 10-30% for a similar period. Upon receiving a small meal indication, the algorithm may increase basal insulin delivery by 5-15% or make more conservative adjustments. Upon receiving an exercise indication with a three-hour lead time, the algorithm may begin reducing basal insulin delivery by 10-20% starting two hours before the indicated exercise time, or may raise the target glucose level from 100 mg/dL to 120-140 mg/dL during the pre-exercise period. The specific adjustments may be personalized based on the user's insulin sensitivity, historical response patterns, and current glucose levels. The algorithm may also adjust the duration of insulin delivery modifications based on the type of activity indicated and the user's historical glucose response to similar activities.

The system may be configured to handle multiple contextual information inputs provided in succession or overlapping time periods. In some aspects, the algorithm may queue multiple inputs and apply their effects cumulatively or according to priority rules. For example, if a user inputs both a meal indication and an exercise indication, the algorithm may initially increase insulin delivery in response to the meal, but then reduce the magnitude or duration of the increase to account for the upcoming exercise. The algorithm may use weighting factors or priority schemes to resolve potentially conflicting inputs, such as giving higher priority to hypoglycemia prevention (exercise input) over hyperglycemia correction (meal input). In some aspects, the algorithm may learn from historical outcomes to optimize its response to combinations of contextual inputs. The system may maintain a queue of pending contextual information inputs and process them according to their temporal relevance and priority, ensuring that the most critical information influences insulin delivery decisions appropriately. When multiple inputs are received within a short time period, the algorithm may analyze the combined effect on predicted glucose levels and adjust insulin delivery parameters to balance competing physiological demands.

The interconnections between modules in the contextual processing system 100 enable bidirectional information flow and coordinated operation. The confirmation handler 108 transmits validated contextual information to the hybrid closed loop 112, which utilizes this information along with integrated data from the context data correlator 122 to control both the insulin adjustment calculator 114 and the target glucose modifier 116. This architecture enables the system to convert simple button press sequences into sophisticated insulin delivery adjustments that account for both current glucose levels and anticipated physiological changes.

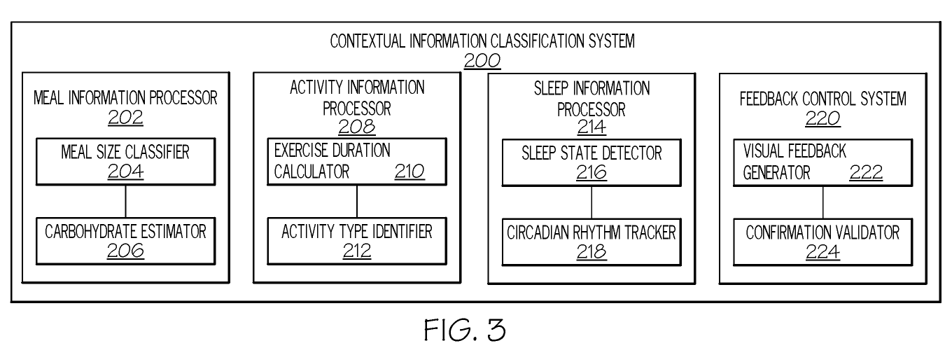

Referring to FIG. 3, a contextual information classification system 200 is illustrated in a block diagram showing the specialized processing modules for different types of contextual information. The contextual information classification system 200 comprises four primary processing modules that work together to interpret, classify, and validate user-provided contextual information before transmission to the hybrid closed loop algorithm. It should be understood that the contextual information classification system 200 shown may be one exemplary modular system, but that the invention may be embodied by any system which performs the functionality described herein.

The meal information processor 202 handles food intake data received from user button press sequences. Within the meal information processor 202, the meal size classifier 204 categorizes meal inputs into different size categories based on the number of button presses received. In some aspects, the meal size classifier 204 may distinguish between small, medium, and large meals, with each category corresponding to a different number of button presses on a scale. The meal information indicates a size of a meal being consumed by the user, wherein the meal information is communicated through a first number of button presses corresponding to small, medium, or large meal sizes. The terms 'first number of button presses' and 'second number of button presses' as used herein refer to different types of contextual information inputs that may be distinguished by the system, rather than indicating a required temporal sequence. The user may provide meal information, physical activity information, or sleep status information in any order and at any time as needed. The scale of meal sizes may be predetermined and programmed into the system, or may be configurable by the user or healthcare provider. In some aspects, a scale of 3 may be used where one button press indicates a small meal, two button presses indicate a medium meal, and three button presses indicate a large meal. In other aspects, different scales may be used, such as a scale of 5 for finer granularity in meal size reporting. The system may store the configured scale and interpret button press counts accordingly. For example, if the scale of meal sizes is 3, the user would press the button three times indicating a large input of food is being eaten. In some aspects, a white LED light may confirm to the software that it is awaiting input, and the user would press the button in one second increments as many inputs as appropriate. The classified meal data is transmitted to the carbohydrate estimator 206, which processes the meal size classification to estimate carbohydrate content for insulin dosing calculations. The carbohydrate estimator 206 may utilize stored user-specific parameters or population-based estimates to convert meal size categories into estimated carbohydrate quantities that can be used by the hybrid closed loop algorithm to calculate appropriate insulin delivery adjustments. At the end of the presses, the pump may confirm with a repeat of the button presses through sound or vibration, and the patient would confirm with a 3-5 second confirmation hold. A 10 second press would cancel the input if necessary.

The activity information processor 208 manages physical activity-related inputs from users. The exercise duration calculator 210 receives button press sequences that indicate timing information for upcoming physical activities and calculates the duration until exercise begins. The physical activity information indicates upcoming exercise by the user, wherein the physical activity information is communicated through a second number of button presses indicating a duration until the exercise begins. In some aspects, the exercise duration calculator 210 may interpret different numbers of button presses as corresponding to different time intervals, with each button press representing one hour until exercise begins. In some aspects, each button press may correspond to one hour until exercise begins, such that one press indicates exercise will begin within approximately one hour (e.g., 30-60 minutes), two presses indicate exercise will begin in approximately two hours (e.g., 60-120 minutes), and three presses indicate exercise will begin in approximately three hours (e.g., 120-180 minutes). The ranges account for the approximate nature of user predictions about exercise timing. For example, if exercise will begin in three hours, the patient presses the button three times. In some aspects, a varying LED light such as blue, white, or red may confirm to the software that it is awaiting input on physical activity information. The calculated duration information is transmitted to the activity type identifier 212, which may classify the specific type of exercise or activity being performed. The activity type identifier 212 may utilize historical data or user-specific settings to determine whether the indicated activity is aerobic exercise, resistance training, or other activity types that may have different effects on glucose levels and insulin requirements. At the end of the presses, the pump may confirm with a repeat of the button presses through sound or vibration, and the patient would confirm with a 3-5 second confirmation hold. A 10 second press would cancel the delivery and exercise indication if necessary. This input allows the algorithm to prevent glucose from going too low during exercise by slowing insulin delivery before exercise, allowing the glucose to rise, or by setting the nominal level of glucose up for a duration of time.

The sleep information processor 214 handles sleep-related contextual data provided by users. The sleep state detector 216 identifies bedtime and wake-up inputs from button press sequences, determining whether the user is indicating the beginning or end of a sleep period. In some aspects, a green LED light may confirm to the software that it is awaiting input on sleep information. In some aspects, the system may use a fixed encoding where one button press always indicates bedtime and two button presses always indicate wake-up time, regardless of the actual time of day. In other aspects, the system may use contextual information such as time of day to interpret the input, or may allow the user to configure which button press pattern corresponds to which sleep state. For one press it may indicate bedtime, while two presses may indicate waking up. The sleep state information is transmitted to the circadian rhythm tracker 218, which monitors sleep patterns and timing information over extended periods. The circadian rhythm tracker 218 may analyze historical sleep data to identify patterns in the user's sleep-wake cycle and may provide this information to the hybrid closed loop algorithm to enable circadian-based adjustments to insulin delivery and target glucose levels. At the end of the presses, the pump may confirm with a repeat of the button presses through sound or vibration, and the patient would confirm with a 3-5 second confirmation hold. A 10 second press would cancel the input if necessary.

The feedback control system 220 manages user interaction and confirmation processes for all types of contextual information. The visual feedback generator 222 receives processed information from the carbohydrate estimator 206, the activity type identifier 212, and the circadian rhythm tracker 218. The visual feedback generator 222 generates visual feedback signals that are transmitted to the user interface to indicate which type of contextual information is being received and to confirm the specific parameters being communicated. In some aspects, the visual feedback generator 222 may generate different visual patterns, colors, or displays to distinguish between meal information, physical activity information, and sleep status information. For example, a white LED light may indicate meal input mode, blue/white/red LED lights may indicate physical activity input mode, and a green LED light may indicate sleep status input mode. The visual feedback signals are transmitted to the confirmation validator 224, which handles user confirmation inputs and validates the contextual information before transmission to the hybrid closed loop algorithm. The confirmation validator 224 requires user confirmation by holding the user input button for a first predetermined time period after receiving the sequence of button presses. The confirmation validator 224 may require the user to hold the button for a predetermined time period of 3-5 seconds to confirm the input, or may detect a longer button hold of approximately 10 seconds to cancel the input if an error was made. In some aspects, if the user holds the button for a duration between the first predetermined time period and the second predetermined time period (e.g., between 5 and 10 seconds), the system may continue to wait until either the button is released or the second predetermined time period is reached. Alternatively, the system may treat any hold duration exceeding the first predetermined time period but less than the second predetermined time period as a confirmation, with only holds exceeding the second predetermined time period triggering cancellation. The system may also provide audible or haptic feedback such as sound or vibration to confirm the number of button presses received.

The connections between components in the contextual information classification system 200 illustrate the flow of processed contextual information through specialized processing pathways. Each type of contextual information follows a dedicated processing pathway that extracts relevant parameters and prepares the data for use by the hybrid closed loop algorithm, while the feedback control system 220 provides a unified interface for user confirmation and validation across all information types.

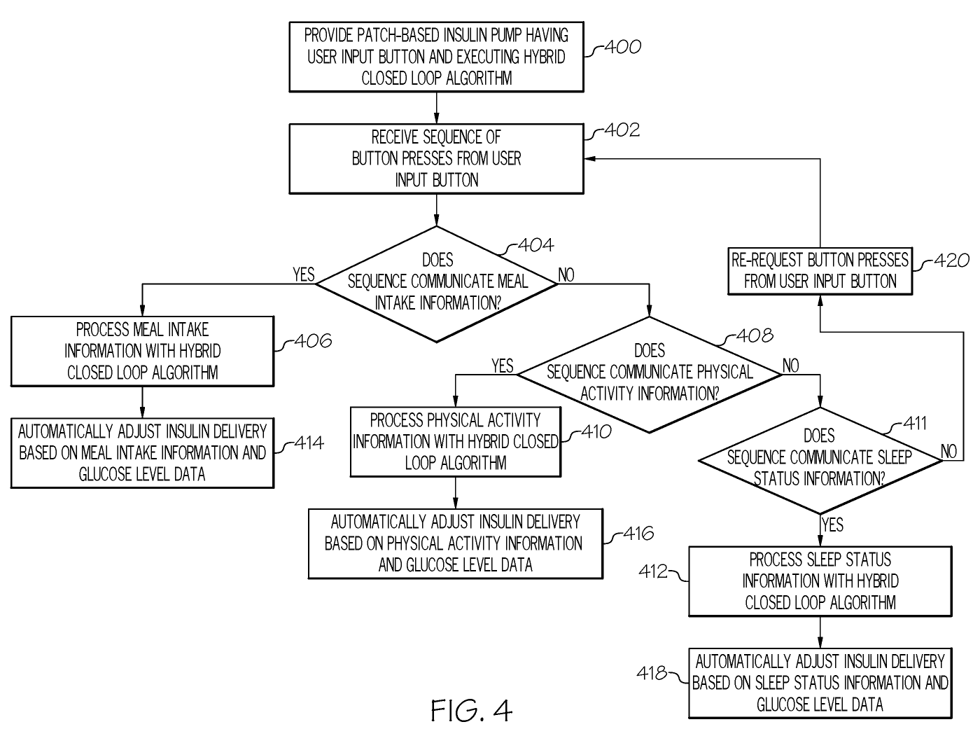

Referring to FIG. 4, a flowchart illustrates an exemplary method of processing multiple types of contextual information in the automated insulin delivery system. It should be understood that while FIG. 4 depicts a particular sequence of determinations, the order of these determinations may vary in different implementations, and the method shown is merely one exemplary embodiment. The method begins at step 400, where a patch-based insulin pump having a user input button and executing a hybrid closed loop algorithm is provided to the user. The system is initialized and ready to receive contextual information inputs from the user.

The process proceeds to step 402, where a sequence of button presses from the user input button is received. The system monitors the button interface for user input and captures the timing and pattern of button presses when the user initiates an input sequence. In some aspects, the button presses may be received in one second increments, with the system analyzing the intervals between presses to determine the type and content of the contextual information being communicated. The received button press sequence is then analyzed to determine what type of contextual information the user is attempting to communicate.

At step 404, a decision point determines whether the sequence communicates meal intake information. The system analyzes the pattern of button presses, including the number of presses and the timing intervals between presses, to determine if the sequence matches a predefined pattern for meal information input. While FIG. 4 illustrates this determination occurring before the physical activity and sleep status determinations, it should be understood that in other embodiments, this determination may occur after one or both of the other determinations, or may occur simultaneously with the other determinations. If the decision at step 404 determines that the sequence communicates meal intake information, the process follows the Yes branch to step 406, where the meal intake information is processed with the hybrid closed loop algorithm. The algorithm interprets the meal size information and calculates appropriate insulin delivery adjustments based on the estimated carbohydrate content of the meal. The process then continues to step 414, where insulin delivery is automatically adjusted based on the meal intake information and glucose level data. The hybrid closed loop algorithm may increase basal insulin delivery rates or calculate a meal bolus to compensate for the anticipated glucose rise from the meal.

If the decision at step 404 determines that the sequence does not communicate meal intake information, the process follows the No branch to step 408, which presents a second decision point asking whether the sequence communicates physical activity information. The system analyzes the button press pattern to determine if it matches predefined patterns for physical activity input. As noted above, in alternative embodiments, this determination may occur before the meal intake determination, after the sleep status determination, or simultaneously with one or both of the other determinations. If the decision at step 408 determines that the sequence communicates physical activity information, the process follows the Yes branch to step 410, where the physical activity information is processed with the hybrid closed loop algorithm. The algorithm interprets the timing information for upcoming exercise and prepares to make proactive adjustments to insulin delivery. The process then continues to step 416, where insulin delivery is automatically adjusted based on the physical activity information and glucose level data. The hybrid closed loop algorithm may reduce basal insulin delivery rates or raise target glucose levels in anticipation of the exercise to reduce the risk of hypoglycemia during physical activity.

If the decision at step 408 determines that the sequence does not communicate physical activity information, the process follows the No branch to step 412, which presents a third decision point asking whether the sequence communicates sleep status information. The system analyzes the button press pattern to determine if it matches predefined patterns for sleep status input. In alternative embodiments, this determination may occur first, second, or simultaneously with the meal intake and physical activity determinations. If the decision at step 412 determines that the sequence communicates sleep status information, the process follows the Yes branch to step 418, where the sleep status information is processed with the hybrid closed loop algorithm. The algorithm interprets whether the user is indicating bedtime or wake-up time and prepares to adjust insulin delivery parameters for the sleep period or waking period. Following step 418, insulin delivery is automatically adjusted based on the sleep status information and glucose level data, with the algorithm potentially modifying target glucose levels or basal rates to optimize overnight glucose control or to accommodate the physiological changes associated with waking.

If the decision at step 412 determines that the sequence does not communicate sleep status information, the process follows the No branch to step 411, where button presses from the user input button are re-requested. The system may provide feedback to the user indicating that the button press sequence was not recognized and prompting the user to try again. Following step 411, the process returns via step 420 to step 402 to again receive a sequence of button presses from the user input button, allowing the user to re-enter the contextual information using a correct button press pattern.

The flowchart demonstrates an exemplary hierarchical decision-making process that systematically categorizes different types of contextual information based on button press sequences, with each type of information leading to appropriate insulin delivery adjustments through the hybrid closed loop algorithm. It should be understood that the sequential order of determinations shown in FIG. 4 is merely illustrative, and any order of determinations is possible so long as the system determines whether a sequence of inputs relates to one or more of meal intake, physical activity, sleep status, and/or other user behaviors. In some embodiments, the system may perform all three determinations simultaneously using parallel processing, pattern matching algorithms, or other techniques that evaluate the button press sequence against multiple predefined patterns concurrently. Additionally, the system may be configured to recognize other types of contextual information beyond meal intake, physical activity, and sleep status, such as stress levels, illness, medication intake, or other behaviors that may affect insulin requirements.

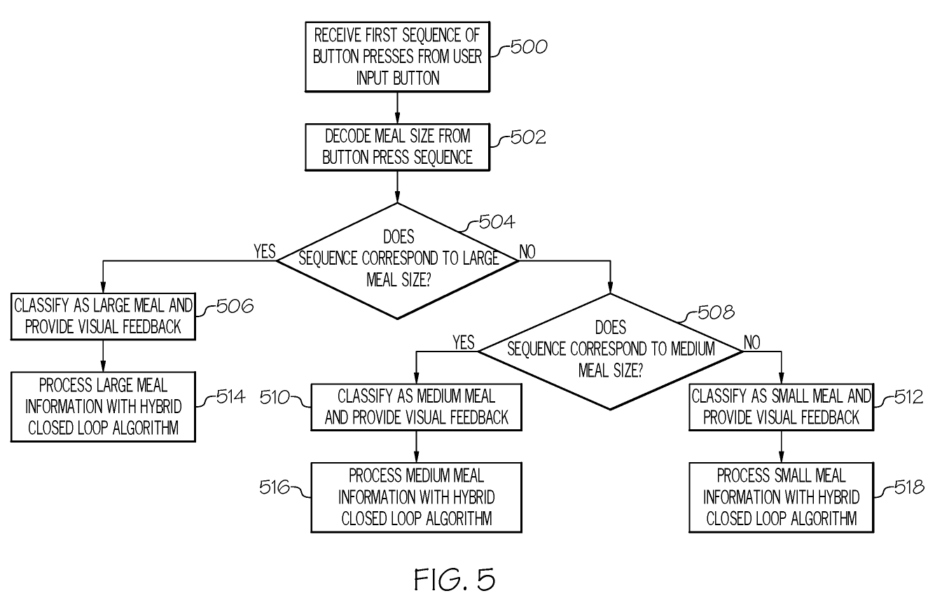

Referring to FIG. 5, a flowchart illustrates an exemplary method of processing meal size information in the automated insulin delivery system. It should be understood that while FIG. 5 depicts a particular sequence of determinations, the order of these determinations may vary in different implementations, and the method shown is merely one exemplary embodiment. The method begins at step 500, where a first sequence of button presses is received from the user input button. The system captures the button press sequence initiated by the user to communicate meal intake information.

The process proceeds to step 502, where the meal size is decoded from the button press sequence. The system analyzes the number of button presses and the timing pattern to determine which meal size category the user is indicating. In some aspects, the decoding process may count the number of discrete button presses within a predetermined time window to classify the meal size.

At step 504, a decision point determines whether the sequence corresponds to a large meal size. The system compares the decoded button press count to a threshold value that distinguishes large meals from other meal sizes. In some aspects, if the scale of meal sizes is 3, three button presses would indicate a large meal. While FIG. 5 illustrates this determination occurring before the medium and small meal determinations, it should be understood that in other embodiments, this determination may occur after one or both of the other determinations, or may occur simultaneously with the other determinations. If the decision at step 504 determines that the sequence corresponds to a large meal size, the process follows the Yes branch to step 506, where the system classifies the input as a large meal and provides visual feedback to the user. The visual feedback may include illuminating a display element such as a white LED light, changing a display color, or presenting a specific visual pattern that confirms the large meal classification. The system may also provide audible or haptic feedback such as sound or vibration repeating the number of button presses. The process then continues to step 514, where the large meal information is processed with the hybrid closed loop algorithm. The algorithm may calculate a larger insulin bolus or make more aggressive adjustments to basal insulin delivery rates to compensate for the anticipated glucose rise from the large meal.

If the decision at step 504 determines that the sequence does not correspond to a large meal size, the process follows the No branch to step 508, which presents a second decision point asking whether the sequence corresponds to a medium meal size. The system compares the decoded button press count to a second threshold value that distinguishes medium meals from small meals. As noted above, in alternative embodiments, this determination may occur before the large meal determination, after the small meal determination, or simultaneously with one or both of the other determinations. If the decision at step 508 determines that the sequence corresponds to a medium meal size, the process follows the Yes branch to step 510, where the system classifies the input as a medium meal and provides visual feedback to the user. The visual feedback confirms the medium meal classification through appropriate display elements or patterns. The process then continues to step 516, where the medium meal information is processed with the hybrid closed loop algorithm. The algorithm calculates insulin delivery adjustments appropriate for a medium-sized meal, which may be intermediate between the adjustments made for large and small meals.

If the decision at step 508 determines that the sequence does not correspond to a medium meal size, the process follows the No branch to step 512, where the system classifies the input as a small meal and provides visual feedback to the user. The visual feedback confirms the small meal classification, indicating to the user that the system has received and interpreted their input correctly. In alternative embodiments, this determination may occur first, second, or simultaneously with the large meal and medium meal determinations. Following step 512, the process continues to step 518, where the small meal information is processed with the hybrid closed loop algorithm. The algorithm calculates insulin delivery adjustments appropriate for a small meal, which may involve more conservative increases to basal insulin delivery or smaller meal boluses compared to larger meal sizes.

The flowchart demonstrates an exemplary hierarchical decision-making process that systematically categorizes meal size information into three distinct categories based on button press sequences, with each category leading to appropriate insulin delivery adjustments through the hybrid closed loop algorithm after providing user feedback confirmation. It should be understood that the sequential order of determinations shown in FIG. 5 is merely illustrative, and any order of determinations is possible so long as the system determines whether a sequence of inputs relates to one or more of large, medium, and small meal sizes. In some embodiments, the system may perform all three determinations simultaneously using parallel processing, pattern matching algorithms, or other techniques that evaluate the button press sequence against multiple predefined patterns concurrently.

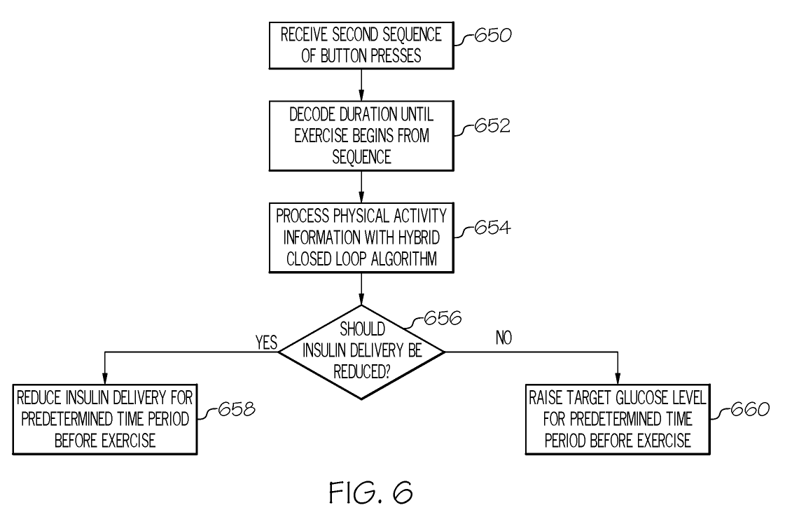

Referring to FIG. 6, a flowchart illustrates a method of processing physical activity information in the automated insulin delivery system. The method begins at step 650, where a second sequence of button presses is received from the user input button. The system captures the button press sequence initiated by the user to communicate information about upcoming physical activity.

The process proceeds to step 652, where the duration until exercise begins is decoded from the received button press sequence. The system analyzes the number of button presses and the timing pattern to determine how much time will elapse before the user begins exercising. In some aspects, each button press may correspond to one hour until exercise begins, such that one press indicates exercise will begin within approximately one hour (e.g., 30-60 minutes), two presses indicate exercise will begin in approximately two hours (e.g., 60-120 minutes), and three presses indicate exercise will begin in approximately three hours (e.g., 120-180 minutes). The ranges account for the approximate nature of user predictions about exercise timing. The decoded duration information provides the hybrid closed loop algorithm with advance notice of the upcoming activity, enabling proactive adjustments to insulin delivery.

Following the decoding step, the process continues to step 654, where the physical activity information is processed with the hybrid closed loop algorithm. The algorithm analyzes the decoded duration information along with current glucose levels, recent insulin delivery history, and other relevant factors to determine appropriate adjustments to insulin delivery parameters. The algorithm may consider factors such as the user's typical glucose response to exercise, current insulin on board, and the time available before exercise begins to calculate optimal adjustments.

At step 656, a decision point determines whether insulin delivery should be reduced based on the processed physical activity information. The hybrid closed loop algorithm evaluates whether reducing insulin delivery is the appropriate strategy for preventing exercise-induced hypoglycemia. This decision may be based on factors such as current glucose levels, the duration until exercise begins, and the user's insulin sensitivity. If the decision at step 656 determines that insulin delivery should be reduced, the process follows the Yes branch to step 658, where insulin delivery is reduced for a predetermined time period before the indicated exercise begins. The reduction in insulin delivery may involve decreasing basal insulin delivery rates to allow glucose levels to rise slightly before exercise, reducing the risk of hypoglycemia during physical activity. The predetermined time period may be calculated based on the decoded duration information and the pharmacokinetics of the insulin being delivered.

If the decision at step 656 determines that insulin delivery should not be reduced, the process follows the No branch to step 660, where the target glucose level is raised for a predetermined time period before the exercise begins. Rather than reducing insulin delivery, the algorithm adjusts the target glucose level upward, which may cause the hybrid closed loop algorithm to deliver less insulin as it works to maintain glucose levels at the higher target. This approach may be preferred in situations where current glucose levels are already elevated or where the user's glucose response to exercise is less predictable. The raised target glucose level provides a buffer against exercise-induced glucose drops while maintaining algorithmic control over insulin delivery.

The flowchart demonstrates the algorithmic decision-making process for managing insulin delivery in anticipation of physical activity, with the system choosing between two alternative strategies—reducing insulin delivery or adjusting target glucose levels—based on the specific circumstances and the processed physical activity information.

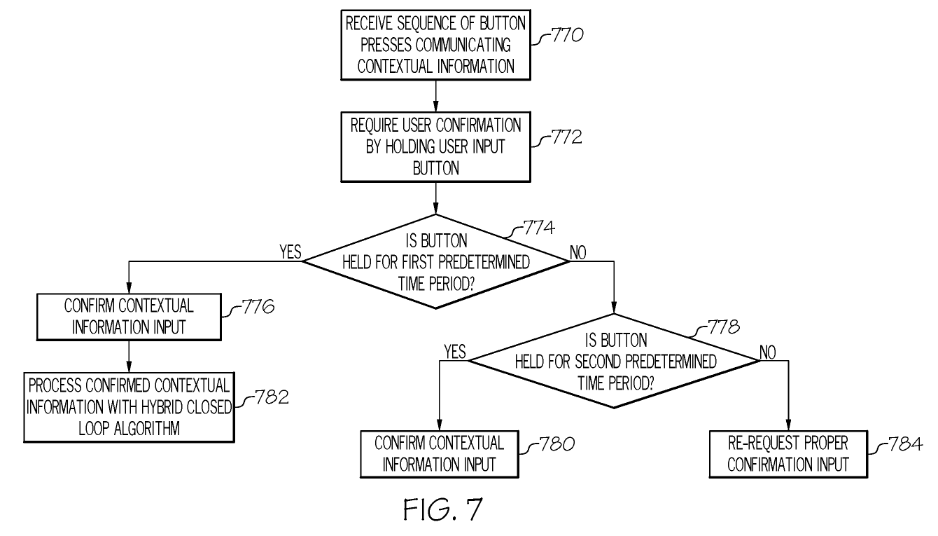

Referring to FIG. 7, a flowchart illustrates a method of processing contextual information with user confirmation in the automated insulin delivery system. It should be understood that while FIG. 7 depicts a particular sequence of determinations, the order of these determinations may vary in different implementations, and the method shown is merely one exemplary embodiment. The method begins at step 770, where a sequence of button presses communicating contextual information is received from the user input button. The system captures the button press sequence that may represent meal information, physical activity information, or sleep status information.

The process proceeds to step 772, where user confirmation is required by holding the user input button. After receiving the initial button press sequence, the system enters a confirmation mode where it monitors for a button hold action from the user. The system may provide visual or audible feedback to indicate that it is waiting for confirmation, prompting the user to hold the button to confirm the input or to take no action if the input was entered in error. The system may also provide instructions or cues to guide the user through the confirmation process.

At step 774, a decision point determines whether the button is held for a first predetermined time period. The system monitors the duration of the button hold and compares it to a first time threshold that indicates confirmation. The first predetermined time period may be set to a duration that is long enough to distinguish intentional confirmation from accidental button contact, but short enough to provide a responsive user experience. In some aspects, the first predetermined time period may be in the range of 3 to 5 seconds. While FIG. 7 illustrates this determination occurring before the second predetermined time period determination, it should be understood that in other embodiments, this determination may occur after the other determination, or may occur simultaneously with the other determination. If the decision at step 774 determines that the button is held for the first predetermined time period, the process follows the Yes branch to step 776, where the contextual information input is confirmed. The system validates the received button press sequence and prepares to transmit the contextual information to the hybrid closed loop algorithm. The process then continues to step 782, where the confirmed contextual information is processed with the hybrid closed loop algorithm. The algorithm receives the validated contextual information and uses it to make appropriate adjustments to insulin delivery parameters based on the type and content of the information provided.

If the decision at step 774 determines that the button is not held for the first predetermined time period, the process follows the No branch to step 778, which presents a second decision point asking whether the button is held for a second predetermined time period. The system continues to monitor the button hold duration and compares it to a second time threshold that indicates cancellation. The second predetermined time period is longer than the first predetermined time period, providing a distinct time window for cancellation actions. In some aspects, the second predetermined time period may be approximately 10 seconds, providing clear separation from the confirmation time period. As noted above, in alternative embodiments, this determination may occur before the first predetermined time period determination, or simultaneously with the first predetermined time period determination. In some aspects, if the user holds the button for a duration between the first predetermined time period and the second predetermined time period (e.g., between 5 and 10 seconds), the system may continue to wait until either the button is released or the second predetermined time period is reached. Alternatively, the system may treat any hold duration exceeding the first predetermined time period but less than the second predetermined time period as a confirmation, with only holds exceeding the second predetermined time period triggering cancellation. If the decision at step 778 determines that the button is held for the second predetermined time period, the process follows the Yes branch to step 780, where the contextual information input is canceled. The system discards the received button press sequence and returns to a ready state, waiting for new input from the user. The cancellation action allows users to correct errors in their input without requiring complex menu navigation or additional button configurations.

If the decision at step 778 determines that the button is not held for the second predetermined time period, the process follows the No branch to step 784, where proper confirmation input is re-requested. The system may provide feedback to the user indicating that the button hold duration was insufficient for either confirmation or cancellation, and prompting the user to try again. In alternative embodiments, this determination may occur first, second, or simultaneously with the first and second predetermined time period determinations. Following step 784, the process returns to step 772 to again require user confirmation by holding the user input button, giving the user another opportunity to either confirm or cancel the contextual information input.

The flowchart demonstrates an exemplary time-based confirmation mechanism that distinguishes between confirmation and cancellation actions based on button hold duration, providing users with intuitive control over the input process while ensuring accurate transmission of contextual information to the hybrid closed loop algorithm. It should be understood that the sequential order of determinations shown in FIG. 7 is merely illustrative, and any order of determinations is possible so long as the system determines whether a button hold duration corresponds to one or more of confirmation, cancellation, and/or re-request of input. In some embodiments, the system may perform all determinations simultaneously using parallel processing, pattern matching algorithms, or other techniques that evaluate the button hold duration against multiple predefined thresholds concurrently.

The flowchart demonstrates a time-based confirmation mechanism that distinguishes between confirmation and cancellation actions based on button hold duration, providing users with intuitive control over the input process while ensuring accurate transmission of contextual information to the hybrid closed loop algorithm.

The disclosed system provides significant advantages over traditional app-based input methods. Rather than requiring users to open an app on their phone, navigate to the input screen, and indicate the desired input of information, the button-based contextual input system allows users to discretely provide information directly through the patch-based insulin pump. This discrete input capability is particularly valuable in social situations where users may prefer not to draw attention to their diabetes management activities. The button on the patch pump system attached to the body enables real-time communication of personalized data without the need for external devices, enhancing user engagement and improving the quality of information available to the hybrid closed loop algorithm.

Beyond the primary contextual variables of meal intake, physical activity, and sleep status, there are many other potential variables that could be communicated through button press sequences to enhance insulin management. Insulin management is a highly individualized aspect of diabetes care, and continuous monitoring and adjustments are essential for optimal diabetes management. The button-based input system would enable users to discretely input personalized data in real-time for a variety of additional contextual factors.

Hormonal changes represent one category of additional contextual variables that significantly impact insulin requirements. Menstrual cycle phases can affect insulin sensitivity and glucose levels, requiring adjustments to insulin delivery. Stress responses trigger hormonal changes that can elevate glucose levels and alter insulin needs. Illness and infection activate immune responses that often increase insulin resistance and require more aggressive insulin dosing. Users could communicate these hormonal factors through dedicated button press sequences, allowing the hybrid closed loop algorithm to anticipate and respond to the associated changes in insulin requirements.

Medications represent another important category of contextual variables. Steroids and other hormones can significantly reduce insulin sensitivity, requiring substantial increases in insulin delivery during treatment periods. Other medications, including some drugs for hypertension, HIV, and psychiatric conditions, can affect glucose metabolism and insulin needs. Users taking these medications could alert the algorithm through button press inputs, enabling appropriate adjustments to insulin delivery parameters during medication periods.

Environmental factors also influence insulin absorption and glucose metabolism. Higher altitudes can affect insulin absorption rates and glucose metabolism through changes in oxygen availability and physiological stress responses. Hot weather can increase blood circulation, speeding up insulin absorption and potentially leading to hypoglycemia if insulin delivery is not adjusted accordingly. Cold weather can decrease peripheral blood flow and slow down insulin absorption, requiring different insulin delivery strategies. Users could communicate these environmental conditions through button press sequences, allowing the algorithm to adjust insulin delivery timing and quantities based on anticipated changes in insulin pharmacokinetics.

The button-based contextual input system thus provides a flexible platform for communicating a wide range of personalized data that affects insulin requirements. By enabling discrete, real-time input of contextual information without requiring external devices or complex menu navigation, the system enhances user engagement and provides the hybrid closed loop algorithm with the information necessary for more precise and individualized insulin delivery control.

The disclosed contextual input system represents a significant advancement over traditional insulin pump interfaces and automated delivery systems. Conventional systems require users to navigate complex menu structures or open external applications to provide contextual information, creating barriers to user engagement and limiting the quality of data available to control algorithms. The inventive button-based input mechanism overcomes these limitations by enabling discrete, real-time communication of personalized activity data through simple button press sequences that can be executed directly on the patch-based insulin pump.

The technical advantages of the disclosed system include improved algorithmic performance through enhanced contextual awareness, allowing the hybrid closed loop algorithm to proactively adjust insulin delivery parameters in anticipation of physiological changes rather than reactively responding to glucose excursions after they occur. This proactive capability may reduce glucose variability and time spent in above-range glucose levels, which currently affects users approximately 30% of the time. The simplified input interface reduces the cognitive burden on users while simultaneously increasing the likelihood of consistent contextual information provision, creating a positive feedback loop that enhances both user experience and clinical outcomes.

The multi-dimensional encoding capability of the system, which enables communication of different types and magnitudes of contextual information through variations in button press sequences, provides a level of specificity and flexibility that was previously achievable only through complex multi-button interfaces or extensive menu navigation. The integrated confirmation and cancellation mechanisms ensure data accuracy while maintaining interface simplicity, addressing a common challenge in medical device design where error prevention often comes at the cost of increased complexity.

The wireless communication architecture enables the system to integrate contextual information with continuous glucose monitoring data and historical pattern analysis, providing the hybrid closed loop algorithm with a comprehensive view of the user's physiological state and anticipated needs. This integration allows for more sophisticated insulin delivery adjustments that account for individual variability in insulin sensitivity, glucose response patterns, and daily activity routines. The discrete nature of the button-based input mechanism also addresses an important psychosocial aspect of diabetes management, allowing users to provide necessary information without drawing attention to their condition in social or professional settings.

A number of implementations have been described. Nevertheless, it will be understood that various modifications may be made without departing from the spirit and scope of the disclosure. Accordingly, other implementations are within the scope of the following claims.

1. An insulin delivery system for diabetes management, comprising:
a patch-based insulin pump configured to deliver insulin to a user;
a user input button disposed on the patch-based insulin pump;
a processor configured to execute a hybrid closed loop algorithm that automatically controls insulin delivery based on glucose levels; and
wherein the user input button is configured to receive user input comprising a sequence of button presses that communicate contextual information about user activities to the hybrid closed loop algorithm, the contextual information being distinct from direct insulin bolus delivery commands.

2. The insulin delivery system of claim 1, further comprising a glucose sensor in communication with the processor configured to provide the processor with glucose level data.

3. The insulin delivery system of claim 2, wherein the processor is a remote computing system in wireless communication with the patch-based insulin pump, the user input button, and the glucose sensor.

4. The insulin delivery system of claim 1, wherein the contextual information comprises meal information indicating a size of a meal being consumed by the user, wherein the meal information is communicated through a first number of button presses corresponding to small, medium, or large meal sizes.

5. The insulin delivery system of claim 1, wherein the contextual information comprises physical activity information indicating upcoming exercise by the user, wherein the physical activity information is communicated through a second number of button presses indicating a duration until the exercise begins.

6. The insulin delivery system of claim 1, wherein the contextual information comprises sleep information indicating bedtime or wake-up time of the user.

7. The insulin delivery system of claim 1, further comprising a confirmation mechanism that provides feedback to the user after receiving the sequence of button presses.

8. A method of managing diabetes using an automated insulin delivery system, comprising:
providing a patch-based insulin pump having a user input button and executing a hybrid closed loop algorithm;
receiving a sequence of button presses from the user input button, wherein the sequence of button presses communicates contextual information about user activities selected from the group consisting of meal intake information, physical activity information, and sleep status information;
processing the contextual information with the hybrid closed loop algorithm; and
automatically adjusting insulin delivery based on the contextual information and glucose level data.

9. The method of claim 8, wherein the meal intake information is communicated through a first sequence of button presses corresponding to small, medium, or large meal sizes.

10. The method of claim 9, further comprising a step of providing visual feedback to confirm receipt of the meal intake information before processing by the hybrid closed loop algorithm.

11. The method of claim 8, wherein the physical activity information is communicated through a second sequence of button presses indicating a duration until exercise begins.

12. The method of claim 11, wherein automatically adjusting insulin delivery comprises reducing insulin delivery or raising a target glucose level for a predetermined time period before the indicated exercise.

13. The method of claim 8, further comprising a step of requiring user confirmation by holding the user input button for a first predetermined time period after receiving the sequence of button presses.

14. The method of claim 13, further comprising a step of canceling the contextual information input when the user input button is held for a second predetermined time period, wherein the second predetermined time period is longer than the first predetermined time period.

15. An insulin delivery system for diabetes management, comprising:
a patch-based insulin pump;
a user input button disposed on the patch-based insulin pump;
a processor configured to execute a hybrid closed loop algorithm that automatically controls insulin delivery based on glucose levels;
wherein the user input button is configured to receive different sequences of button presses corresponding to different types of contextual information about user activities; and
wherein the processor is configured to modify operation of the hybrid closed loop algorithm based on the contextual information communicated through the different sequences of button presses.

16. The insulin delivery system of claim 15, wherein the different types of contextual information comprise meal intake information, physical activity information, and sleep status information.

17. The insulin delivery system of claim 16, further comprising a feedback mechanism that provides visual feedback to indicate which type of contextual information is being received.

18. The insulin delivery system of claim 15, further comprising a confirmation mechanism that requires the user to hold the user input button for a first predetermined time period to confirm the contextual information being communicated to the hybrid closed loop algorithm, and wherein the confirmation mechanism is configured to cancel the contextual information input when the user input button is held for a second predetermined time period, wherein the second predetermined time period is longer than the first predetermined time period.

19. The insulin delivery system of claim 15, wherein the processor is configured to distinguish between the different sequences of button presses based on timing intervals between individual button presses within each sequence.

20. The insulin delivery system of claim 15, wherein the hybrid closed loop algorithm is configured to adjust at least one of basal insulin delivery rate or target glucose level in response to receiving the contextual information.
